(19) United States
(12) Patent Application Publication
(10) Pub. No.: US 2023/0414167 A1
(43) Pub. Date: Dec. 28, 2023

(54) POSTURAL ASSESSMENT DEVICE AND THERAPEUTIC METHOD

(71) Applicant: Steve Veltman, Oceanside, CA (US)

(72) Inventor: Steve Veltman, Oceanside, CA (US)

(21) Appl. No.: 18/086,068

(22) Filed: Dec. 21, 2022

Related U.S. Application Data
(60) Provisional application No. 63/292,749, filed on Dec. 22, 2021.

(51) Int. Cl. A61B 5/00; A61B 5/107

(52) U.S. Cl. CPC A61B 5/4561; A61B 5/1077; A61B 5/0064

(57) ABSTRACT

A physical surface modeling the topography of an area of interest on a patient's body is prepared from a scan of the patient when the patient is postured in the best approximation of a neutral spine alignment. The surface is a modified model of a negative image of the topography, capturing the best current configuration of the patient's postural alignment and related superficial anatomy. The device enables comparison of the configuration of a patient's posture and related superficial anatomy at a given time with the topography of the surface. Thereby, this surface can be used by the therapist and patient as a template for assessment of postural conformity, range of motion and joint limitations of extremities, core function, and other superficial bilateral deviations of the area of interest.

### CROSS-REFERENCE TO RELATED APPLICATION

This application claims the benefit of U.S. provisional patent application No. 63/292,749, filed Dec. 22, 2021, incorporated herein by reference.

### BACKGROUND OF THE INVENTION

### Field of the Invention

This specification relates to a non-invasive device for mapping the best neutral posture configuration of a patient's spine and proximal superficial anatomy. This specification further relates to methods of use of the device for identification and therapeutic treatment of functional range of motion limitations and joint restriction of extremities, minor postural misalignment, quality of diaphragm expansion and assessment of core function.

### Description of the Related Art

Patients commonly seek physical therapy for maladies that are not necessarily the result of injury but rather extremity restrictions resulting in postural misalignment. Symptoms may include muscle stiffness, joint pain, contractures, numbness and rapid fatigue of working muscles, among other pains and inefficiencies. Compensation for these symptoms can lead to deviations in the axial skeletal system, affecting functional posture, with resultant back ache, shallowness of breathing and further overall inefficiency in mechanical function.

The related art has focused principally on assessment and therapies related to spinal alignment. In such art, the therapist makes overall assessment of the spinal column and proximal anatomy for spinal analysis, postural evaluation, and measurement of vertebral joint range of motion. Further, administration of therapies such as spinal manipulation typically involve monitoring the patient's spinal and related posterior anatomical configurations during treatment.

Various means have been employed to assess a patient's spinal configuration. Invasive means, such as X-Ray, computer aided tomography and magnetic resonance imaging, are often used to obtain images of patient's spines. Such invasive means, however, are subject to several limitations. The equipment employed is generally not amenable to obtaining images of the patient in a posture, such as standing or half-standing, that is most suitable for the physical therapist's needs. In addition, X-Ray machinery, CAT scanners and MRI equipment require a substantial capital outlay. Furthermore, in general such equipment is not suitable for monitoring the patient as the patient receives therapy. Yet further, some invasive means, in particular X-ray and CAT scanning, expose the patient to potentially harmful ionizing radiation.

Non-invasive means are also used to assess spinal configuration. Among these are the Metrecom skeletal analysis system, employing a 3-dimensional digitizer in communication with a computer system, as described in U.S. Pat. No. 4,760,851 to Fraser et al. By way of linkage joints and rotational transducers, the digitizer obtains configuration data from the area of interest on the patient, the data then processed by the computer system to derive a mathematical model of the relevant skeletal anatomy. A significant limitation of this technology is that it can produce only a snapshot of the skeletal configuration at one point in time. In its present form, it cannot be used in a dynamic situation in which assessment and treatment are to take place dynamically, with extremity movement of the patient in various positions.

Some other non-invasive approaches to spinal assessment involve use of the Flexicurve ruler, from Staedtler Mars GmbH & Co. of Nurnberg, Germany. Exemplary of such approaches, U.S. Pat. No. 5,582,186 to Wiegand describes a complex system of measurement and calculation used to derive spinal stress unit values for initial spinal assessment and evaluation of therapeutic progress over time. A major limitation of this approach lies in the large number of measurements and calculations required, presenting barriers to dynamic administration as well as the likelihood of accumulated measurement errors leading to incorrect results.

Spinal assessment alone, however, really provides only an indirect measure of underlying problems. When observing human movement it is clear that our extremities are the workhorse of our kinetic chain. Repetitive movement of extremities combined with limitations in functional range of motion can lead to any number of the forementioned symptoms. As a result, these limitations often lead to deviations in the axial skeletal system through compensation, negatively affecting optimal posture while de-stabilizing the axial skeleton foundation. This leads to a postural template that further exacerbates original symptoms and may lead to back or extremity pain, shallowness of breath, loss of core function and basic movement competency, creating overall inefficiency throughout the entire kinetic chain.

What is needed is an effective, non-invasive approach to postural assessment addresses underlying problems more directly. What is needed further is an approach that does not require complex and expensive equipment. What is needed yet further is such an approach providing a template suitable for improving functional range of motion of extremities. Yet further, what is needed is such an approach that can be applied dynamically in the course of therapy. Further still, what is needed further is an approach that is simple to administer and customized for the patient.

### SUMMARY OF THE INVENTION

Embodiments of this invention comprise a physical surface modeling the topography of an area of interest on the patient's body, prepared from a scan of the patient when the patient is postured in the best approximation of a neutral spine alignment. The surface is a modified model of a negative image of the topography, capturing the best current configuration of the patient's postural alignment and related superficial anatomy. The device enables comparison of the configuration of a patient's posture and related superficial anatomy at a given time with the topography of the surface. Thereby, this surface can be used by the therapist and patient as a template for assessment of postural conformity, range of motion and joint limitations of extremities, core function, quality of diaphragm expansion as well as other superficial bilateral deviations of the area of interest, statically or during passive, active, active-assisted, and loaded extremity movements, to determine a course of necessary physical therapy. During therapy, conformance or deviation between the patient's configuration and the configuration captured by the modified model is used to guide therapeutic progress.

Embodiments of the invention can based upon negative image surfaces prepared of the posterior, anterior or lateral views of a patient's superficial anatomy from top of cranium to base of rump which may similarly be used to assess a patient's condition, determine a needed course of physical therapy, and provide a template for treatment.

### BRIEF DESCRIPTION OF THE DRAWINGS

Objects of the present invention as well as advantages, features and characteristics, in addition to methods of operation, function of related elements of structure, and the combination of parts and economies of manufacture, will become apparent upon consideration of the following description and claims with reference to the accompanying drawings, all of which form a part of this specification, wherein like reference numerals designate corresponding parts in the various figures, and wherein:

FIG. 1 shows a patient standing in a best approximation of a neutral spine configuration for an embodiment of the invention;

FIG. 2 shows a patient in a three-quarter sit-stand position mode to approximate a neutral spine configuration for an embodiment of the invention;

FIG. 3 represents a patient scanned by way of a hand-held scanner while standing postured in a best approximation of a neutral spine for an embodiment of the invention;

FIG. 4 illustrates a surface created in embodiment of the invention;

FIG. 5 illustrates a surface created in another embodiment of the invention;

FIG. 6 depicts a supine patient engaged with a surface such as that depicted in FIG. 4;

FIG. 7 depicts a surface such as that depicted in FIG. 4, overlain with a network of pressure sensors;

FIG. 8 depicts the use of a surface such as that depicted in FIG. 7 by a supine patient, resulting in a surface pressure map of the area of interest according to an embodiment of the invention; and

FIG. 9 depicts a segmented, articulated surface created in another embodiment of the invention.

### DETAILED DESCRIPTION OF THE INVENTION

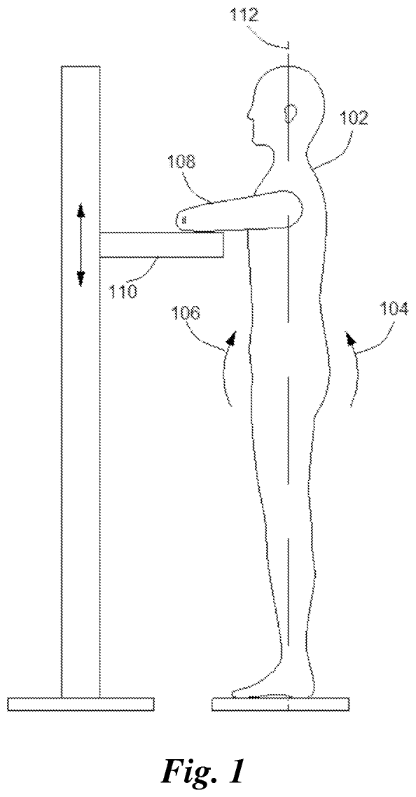

Embodiments of the invention start with a scan of a patient postured in a best approximation of a neutral spine alignment. A therapist can direct a patient in achieving such a posture. One manner of doing so is illustrated in reference to FIG. 1. From a lateral perspective, patient 102 is directed to stand and engage transversus abdominis, gently drawing belly button towards spine. Because a neutral spine is possible only when the pelvis is neutral, patient 102 is directed to rotate the pelvis anteriorly 104 or posteriorly 106, to vertically align the anterior superior iliac spines with the pubic symphysis. Patient 102 is then is instructed to raise both arms forward to parallel, bending elbows at 90° and cross forearms, as illustrated 108. In embodiments, an arm support device 110 may be provided and adjusted to patient height for comfort and reduction of body sway during scanning. Next is plumbline assessment of anatomical landmarks. Plumbline 112 is hung in the lateral view of patient 102. Patient 102 stands next to plumbline with front of lateral malleolus in line with plumbline 112. Patient 102 is then instructed to attempt to align, following anatomical landmarks to the best of their abilities. To do so, patient 102 follows plumbline 112 vertically, slightly anterior to midline through the knee, through greater trochanter, midway through trunk, through shoulder joint (mid acromion), through bodies of cervical vertebrae, finishing through mastoid process of skull.

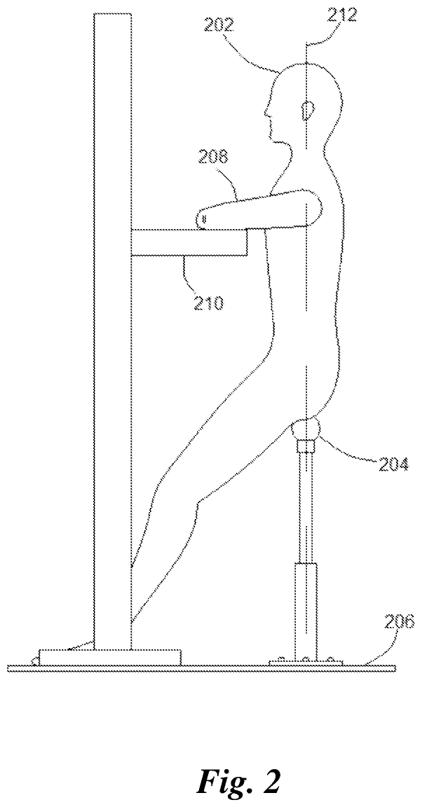

If patient cannot achieve a neutral pelvis without unwanted distortion throughout spine or is suffering postural deviations of the lower limbs, the patient will be directed to the three-quarter sit stand position model in attempt to reduce deviations and improve postural position for scanning area. One manner of doing so is illustrated in reference to FIG. 2. Seat apparatus 204 supports patient 202, whose feet rest on non-slip surface 206. Patient 202 crosses forearms 208 and rests them on adjustable arm support device 210. The height of seat 204 is adjustable so as to allow the desired alignment, illustrated by plumbline 212.

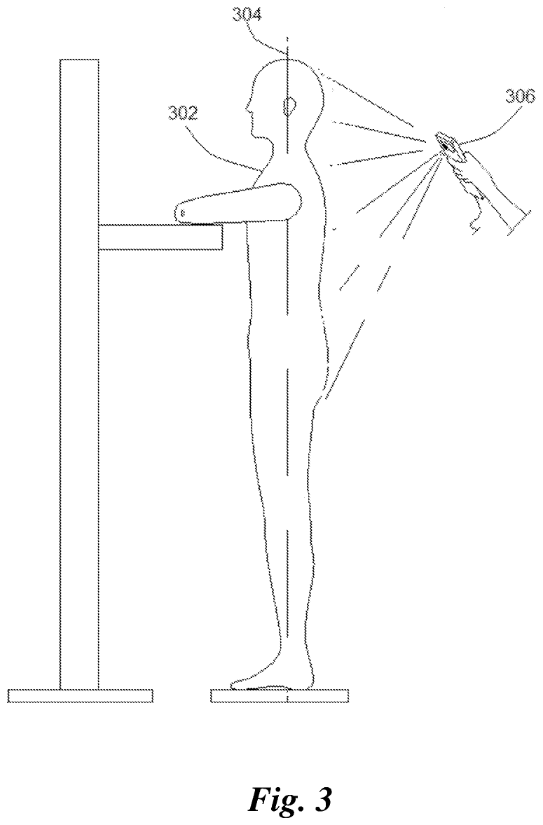

In any case, embodiments of the invention require a scan of the topology of the area of interest of the individual patient's superficial anatomy with the patient postured in a best approximation of a neutral spine alignment. Turning to FIG. 3, illustrated is an embodiment in which a patient 302 is scanned by way of a hand-held scanner 306 while standing postured in a best approximation of a neutral spine, approximating a neutral pelvis, aligning anatomy with plumbline 304 as in FIG. 1. An exemplary embodiment of the invention utilizes the EVA hand-held scanner produced by Artec 3D of Luxembourg, with U.S. facility in Santa Clara, California, employing structured light technology for scanning surfaces.

Scanning is performed to gather a modified superficial topographical image of patient's best approximation of a neutral spine alignment from top of cranium to base of rump, of either anterior, posterior or lateral anatomical views as needed for assessment and therapeutic purposes. Because the anatomical area of interest is determined by the therapeutic requirements as well as the anatomical development of the individual patient at the time of treatment, the scanned area can vary widely, with typical dimensions from 27 to 70 centimeters in width and 57 to 126 centimeters in length. Further, specific patient needs may require scanning smaller or larger areas than are typical. In embodiments depicted herein, the subject area is on the posterior side of the patient while the patient is in a best approximation of a neutral spine posture. Other embodiments may have different subject areas and the posture of the patient for scanning may be adjusted as required. In any case, the scan is used in the various embodiments to create a device, described in more detail below, based upon the topography of the subject area when the patient is in a best approximation of a desired or ideal posture.

Embodiments may process and store the topological data obtained from scanning in one of many formats used by those of skill in the 3-D modeling arts, including 3-D mesh or CAD files. Embodiments then manipulate this data by algorithmic methods well-known to those in the computational arts to define a model of the topography of the patient's subject area. This model may be modified by methods known to those in the computational arts as needed for the application of the invention. The model thus resulting is used to create a physical surface that reflects a negative image of the subject area in the desired posture.

In some embodiments, the physical surface may be created from a model of the subject area that is modified so that it defines displacement lying in the longitudinal plane of patient but disregards displacement along the horizontal plane. In some embodiments, the model of the surface area is modified by smoothing or flattening topographic protrusions due to skeletal features, such as from shoulder blades or vertebrae. In some other embodiments, the model used to create the physical surface may be modified to provide channels accommodating such protrusions. Further, some embodiments may employ a model modified to provide channels for greater range of movement of extremities during assessment and therapy.

In any case, the surface that is created from the modified model of the topography of the patient's subject area captures the postural alignment and related superficial anatomy of the patient in a desired or ideal posture.

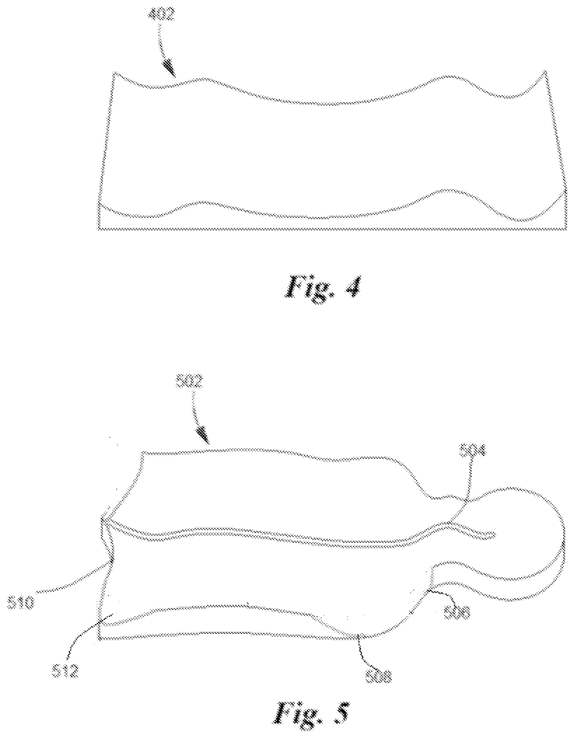

FIG. 4 illustrates a surface 402 created in embodiment of the invention. Surface 402 is the negative image of the subject area topography modified by disregarding displacement along the horizontal plane as described above. Embodiments produce surface 402 as a rigid article by employing computer-aided manufacturing operating on the model developed from the scanned data. Well known to those of skill in the art, such manufacturing processes include machining, either to produce finished articles or molds for finished articles, extrusion, and 3D printing or additive manufacturing. In embodiments employing 3D printing, the article may be fabricated from thermoplastic material common in that technology. Alternatively, molded articles may be fabricated from any suitably rigid substrate material suitable for injection or vacuum molding, as the case may be. It is desirable that the finished surface be relatively light in weight and only sufficiently thick to maintain its rigidity and durability in use, as described below.

FIG. 5 depicts another form of surface. Surface 502 comprises a channel 504 to accommodate spinal process, sometimes prominent particularly in female patients. Surface 502 further comprises shoulder flexion channels (left shoulder flexion channel 506 labeled) and shoulder extension channels (left shoulder extension channel 508 labeled). Yet further, surface 502 comprises hip extension channels (hip extension channel 510 labeled) and hip abduction channels (left hip abduction channel 512 labeled). The extension and flexion channels enable an extended range for assessment of functional movement of extremities and treatment of related issues.

Embodiments use a rigid surface created in accordance with this invention specifically for the individual patient as a template in assessment and therapy directed to functional movement, including but not limited to range of motion of extremities, joint restrictions, core function and quality of diaphragm expansion. By way of illustration, FIG. 6 depicts a patient 602 in supine position on a template surface 604 prepared for the patient according to the teachings of this invention, such as exemplified by surfaces depicted in FIG. 4 or FIG. 5. Through the patient's mechano-receptors, the patient 602 can feel the varied pressure of different portions of their anatomy against the template surface 604 as they breathe and move extremities. The sensory feedback thus obtained enables the patient to compare their current posture with the posture captured in a scan of their best approximation of a neutral spine, thereby aiding the patient in noting deviation from and maintaining adherence to their best neutral spine during therapeutic progress with or without therapist's guidance.

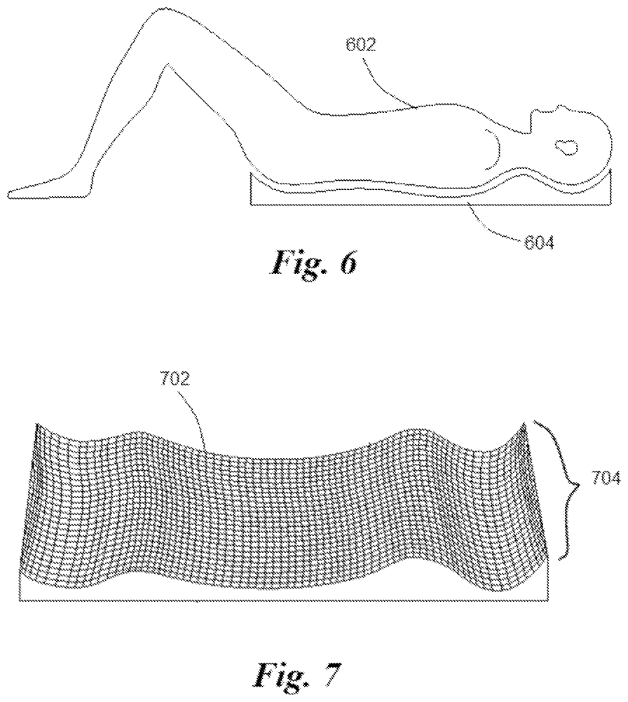

Embodiments of the invention may employ a means of obtaining a real-time pressure map of the patient's subject anatomy during therapy. Illustrated in FIG. 7 is a surface 702 such as depicted in FIG. 4, overlain by a web of analog pressure sensors 704, such the Body Pressure Management System produced by Tekscan, Inc. of Norwood, Massachusetts. Pressure sensors 704 provide data of the varied pressure of a subject's body over surface 702, which can then be used to provide a real-time image of the patient's configuration during therapy. In embodiments, the web of pressure sensors 704 may be affixed to a specific patient's surface 702. In other embodiments, web of sensors 704 may simply be temporarily overlain on a patient's surface as needed for a given therapy session, the web of sensors 704 then reused in sessions with subsequent patients.

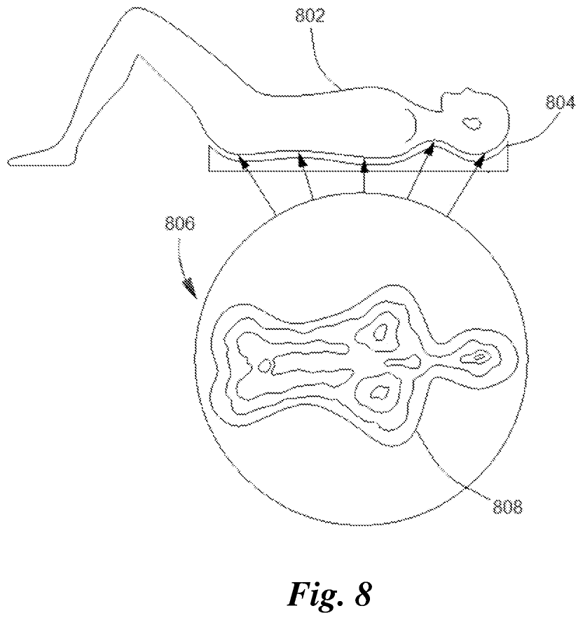

Turning to FIG. 8, illustrated is an embodiment in which a surface equipped with analog sensors is employed to generate a computer image of the patient's current superficial anatomical configuration in the area of interest. Here, patient 802 is again depicted in a supine position. Surface 804, with a web of analog pressure sensors as described in relation to FIG. 7, provides data for a computer to generate a real-time image 806 depiction of a map 808 corresponding to contact and the variation in pressure between the patient and the surface over the subject area. As is well known in the computer visualization arts, map 808 may designate areas sensing differing pressures by different colors. In any case, substantial use of this visual information may be made for dynamic assessment of the patient's postural adherence and related functional range of motion and joint limitations of extremities, facilitating therapeutic correction of extremity and postural deficiencies by enabling patient and therapist to manipulate the patient's anatomy to conform with the individual patient's best postural configuration in real-time. In embodiments, mounting a display so that the patient may view the real-time image, such as directly above a supine patient, facilitates the patient's self-assessment and therapeutic progress.

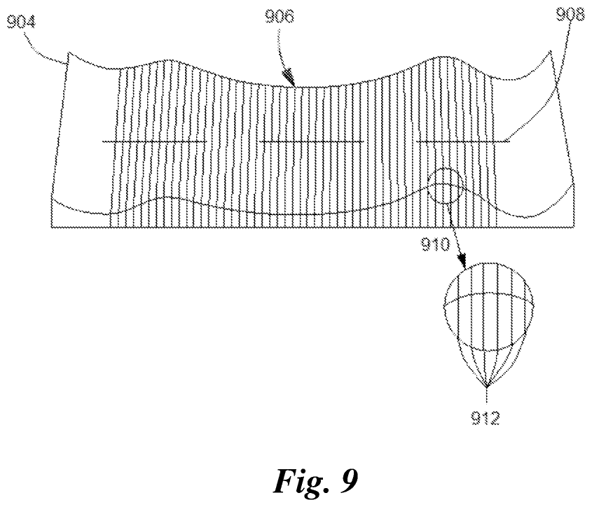

FIG. 9 depicts a different apparatus embodying the invention for obtaining real-time data for assessment and therapy, still based upon a model of the individual patient's anatomy, in embodiments using a best approximation of the patient's neutral spine alignment or other desired postural configuration of the area of interest. In this embodiment, the surface 904 is comprised of a plurality 906 of rigid segments, each segment rotatably disposed on an axis 908 running along a line corresponding to the medial line of the patient's anatomy. A close-up view 910 of some of the segments 912 is illustrated. Each segment may rotate plus or minus a limited angle from the neutral position. In embodiments, the displacement of each segment from the neutral position is obtained as real time data by means, such as potentiometers, well known to those in the art of electronic sensors. The segments are fashioned from the scanned model of the patient's superficial anatomy, so that when all segments are in the neutral position, the surface 904 formed thereby corresponds to the negative image of the patient's superficial anatomy obtained in scanning. In use, a patient engages with surface 904 as described previously. Real time data of the rotation of the individual segments as displaced by the patient in the transverse plane provides an indication of limitations and vertebral rotation and displacement of the patient's posture during therapy and may be used by the patient or the therapist as described above for assessment and treatment of the conditions of interest.

Embodiments of the invention thereby empower patient and therapist by providing a personalized means to address maladies resulting from the patient's postural misalignment, focusing therapeutic conformity of the patient's posture with an idealized, neutral spine configuration specific to that patient, enabling both the patient's postural realignment and reestablishment of basic movement competency of patient's extremities and overall kinetic chain.

While the invention has been described with a certain degree of particularity, it should be recognized that elements thereof may be altered by persons skilled in the art without departing from the spirit and scope of the invention. For example, while embodiments depicted herein illustrate the use of a surface horizontally oriented for a supine patient, other embodiments may employ surfaces in other orientations for patients in different positions. For example, some embodiments may employ a surface oriented vertically or otherwise. In some embodiments, the surface may be provided with a means allowing adjustable change of the orientation of the surface for use from horizontal to vertical and angles in between. Such means may require foot support to provide grounding to keep the patient in place. Foot support in embodiments may further enable pressure mapping of the foot to assess bilateral deviations of standing pressures. In further example, embodiments may be directed to assessments and therapies for the patient in a prone or a lateral position. Yet further, some embodiments may have additional features directed to specific patient health or comfort needs, such as hypoallergenic material or coating for areas of the surface in contact with patient skin, or padding sensitive parts of the patient's body.

As will be understood by those in the art, embodiments may require additional elements to facilitate functionality. For example, embodiments along the lines of the surface depicted in FIG. 5, allowing for additional flexion and extension of upper extremities along with extension and abduction on lower extremities, may require external elements such as stirrups and support surfaces for extremities to enable stabilization and assistance in space for effective therapeutic use of the device. The device may employ active reach assist for upper extremities along with monitoring mechanisms for both upper and lower extremities. Further, as discussed earlier in relation to FIG. 8 but equally applicable in relation to FIG. 9, in those embodiments providing sensor data that enables display of a real-time image of the conformance of the patient's posture to the ideal, mounting a device such as a monitor or tablet to show the image display to the patient will facilitate the patient's self-assessment and therapy.

Accordingly, the present invention is not intended to be limited to the specific forms set forth in this specification, but on the contrary, it is intended to cover such alternatives, modifications and equivalents as can be reasonably included within the scope of the invention. The invention is limited only by the following claims and their equivalents.

I claim:

1. A device for postural assessment and therapy of a patient, made by:
posturing the patient in a best approximation of a neutral spine configuration;
scanning a subject area of superficial anatomy of the patient to obtain a scan dataset;
processing the scan dataset to obtain a topological model of the subject area of superficial anatomy;
modifying the topological model to obtain a mathematical surface model; and
applying computer-aided manufacture to the mathematical surface model to produce a physical surface that is a negative image of the mathematical surface model.

2. The device for postural assessment and therapy of a patient according to claim 1, wherein modifying the topological model to obtain the mathematical surface model comprises disregarding displacement along a horizontal plane.

3. The device for postural assessment and therapy of a patient according to claim 1, wherein modifying the topological model to obtain the mathematical surface model comprises flattening topographic maxima corresponding to protrusions due to skeletal features in the subject area of superficial anatomy.

4. The device for postural assessment and therapy of a patient according to claim 1, wherein modifying the topological model to obtain the mathematical surface model comprises adding channels to correspond to protrusions due to skeletal features on the subject area of superficial anatomy.

5. The device for postural assessment and therapy of a patient according to claim 1, wherein modifying the topological model to obtain the mathematical surface model comprises adding channels corresponding to areas of extremity flexion and extension on the subject area of superficial anatomy.

6. A device for computer-aided postural assessment and therapy of a patient, made by:
posturing the patient in a best approximation of a neutral spine configuration;
scanning a subject area of superficial anatomy of the patient to obtain a scan dataset;
processing the scan dataset to obtain a topological model of the subject area of superficial anatomy;
modifying the topological model to obtain a mathematical surface model;
applying computer-aided manufacture to the mathematical surface model to produce a physical surface that is a negative image of the mathematical surface model; and
overlaying the physical surface with a network of pressure sensors, the network of pressure sensors configured to provide a stream of pressure sensor data to a computer.

7. A device for computer-aided postural assessment and therapy of a patient, comprising:
a sequence of planar segments, each planar segment disposed on a medial line axis allowing rotational displacement from a neutral position, each planar segment further having an upper edge configuration, the upper edge configuration of the sequence of planar segments forming a surface such that, when all planar segments are in the neutral position, the surface conforms to a negative image of a modified topology of a subject area of superficial anatomy of the patient in neutral spine posture, the patient having a patient medial line, the medial line axis aligned to correspond to the patient medial line; and
a set of sensors, each sensor assigned to a planar segment and transmitting, to a computer, a signal corresponding to the rotational displacement of the planar segment from the neutral position.
